SEQUENCE LISTING (9 sequences)

SEQ ID NO: 1 - DNA, 1135 bp, Manihot esculenta Crantz
ggtgatgagaagagagactatttcgttgacaaaaagaaacgataaggatatttaatagatatgaaaaaagataaagacattttaatatatttatgaaaatatagggagggacgatttcgttgacggaaagaaacgatgaggagagactatttcattgacggaaagaaatgatgagaagggactatttcgttgacggaaagaaacgatgaggatggactatttcgttaacggaaagaaacgataaggacgttttaatagatatgagaaaagataaggactatttcattgacggaaagaaacataaggacgttttactagatatgaaaaagataaaaacgttttaatagatttttgaaaatatatggactaacttgttaataatagtaatatccaggactaaataaggggttttaattgagggtaaaattggatttttaaacattttctctagcctcctttatttaattttaatgaaaaagaggacggaaggactatttcgttgacggaaaaaaaatataaggacgttttaataggtttctaaaaatatagggactgacttgttaataatgacaataatcagggactaaattgtaaatctcccaatatatatttactatttagaaagcattaatatattatatattttcacagttgataataattgatgacgaagaaatctcatggatctagctacaagatcaacttgtttaacattagtatcaaccattttgccatttctctttgatttcagtgagatgaggggtcaaatcccaagattcctctttcttaagtgctcccacctcgttctctttcatacatgaacttctggcccttcaattctctatataagccactcttattcatcctctctctgcaccatctctctctttctgtctctccttcctgtttgcttctcagctttattttttagtttctatttccttggctgactatggctactgctgaggtaacccatcaatcatttcttgttaagctttgattcaggttcttgattttaattattgatctcattagtttcagcagctttacatgataatgaaagaattttatcttaaagatcttttgatgaattttgattttaggtagtaacagcacagactgcacttcctgagga

SEQ ID NO: 2 - DNA, 10 bp, Artificial Sequence
  Synthetic polynucleotide
gccacctcat

SEQ ID NO: 3 - DNA, 26 bp, Artificial Sequence
  Synthetic polynucleotide
actggctctgcttccttgggctcttc

SEQ ID NO: 4 - DNA, 26 bp, Artificial Sequence
  Synthetic polynucleotide
tcctcaggaagtgcagtctgtgctgt

SEQ ID NO: 5 - DNA, 26 bp, Artificial Sequence
  Synthetic polynucleotide
gctgatgatgctccggctgaagtagc

SEQ ID NO: 6 - DNA, 31 bp, Artificial Sequence
  Synthetic polynucleotide
ggtgatgagaagagagactatttcgttgaca

SEQ ID NO: 7 - DNA, 27 bp, Artificial Sequence
  Synthetic polynucleotide
tacctcagcagtagccatagtcagcca

SEQ ID NO: 8 - DNA, 33 bp, Artificial Sequence
  Synthetic polynucleotide
agggatccggtgatgagaagagagactatttcg

SEQ ID NO: 9 - DNA, 20 bp, Artificial Sequence
  Synthetic polynucleotide
cagtagccgatggtcagcca

(19) United States
(12) Patent Application Publication
(10) Pub. No.: US 2013/0125261 A1
(43) Pub. Date: May 16, 2013

(54) DNA SEQUENCE CONTAINING THE PROMOTER REGION AND REGULATORELEMENTS OF THE MEC1 GENE, EXPRESSED IN CASSAVA ROOTS, FOR USE IN GENETIC IMPROVEMENT PROGRAMS

(71) Applicants: Cláudia Regina Batista De Souza, Para (BR); Francisco José Lima Aragão, Brasilia (BR); Edith Cibelle De Oliveira Moreira, Belem (BR); Soelange Bezerra Nascimento, Ananindeua (BR); Luiz Joaquim Castelo Blanco Carvalho, Sobradinho (BR)

(72) Inventors: Cláudia Regina Batista De Souza, Para (BR); Francisco José Lima Aragão, Brasilia (BR); Edith Cibelle De Oliveira Moreira, Belem (BR); Soelange Bezerra Nascimento, Ananindeua (BR); Luiz Joaquim Castelo Blanco Carvalho, Sobradinho (BR)

(73) Assignees: Universidad Federal do Para, Para (BR); Embrapa-Empresa Brasileira de Pesquisa Agropecuari, Brasilia (BR)

(21) Appl. No.: 13/636,959

(22) PCT Filed: Mar. 24, 2011
(86) PCT No.: PCT/BR11/00074

§ 371 Date: Jan. 2, 2013

(30) Foreign Application Priority Data
Mar. 24, 2010 (BR) 000040/2010

(51) Int. Cl. C12N 15/82

(52) U.S. Cl. CPC C12N 15/8227
USPC 800/287; 536/24.1; 435/320.1; 435/419; 435/412; 435/414; 435/411; 435/417; 435/416; 435/415; 800/298; 800/278

(57) ABSTRACT

The present invention refers to a promoter and/or specific regulatory regions for the expression of genes of interest in roots. The invention further describes DNA constructs containing the polynucleotide of the invention operatively linked to a heterologous and/endogenous gene. Besides, the invention refers to the use of these constructions in the form of expression vectors, recombinant vectors and in plants, plant cells or transgenic protoplasts. The invention further describes a method employing such constructs containing the polynucleotide of the invention for the production of plants, plant cells or transgenic protoplasts. Thus, the expression of the transgene only in the part of interest enables the accumulation of the exogenous transcript only in the root, favoring the implementation of strategies that aim at increasing the aggregated value, the generation of cultivars more adapted to environmental stress, to pathogens and pests, agrochemicals, besides plants with a high nutritional value and high therapeutic value. In addition to these advantages, the present invention is a new alternative to expression systems in vegetable organisms and may be used for generating new cultivars and improvement programs.

### FIELD OF THE INVENTION

The present invention relates to the field of biotechnology. More specifically, the invention relates to a promoter and to regulatory regions for expression of molecules of interest in plant roots. More specifically, the present invention relates to a promoter and to manioc (Manihot esculenta Crantz) Mec1 gene regulatory regions. The invention further describes DNA constructs containing the promoter of the invention operatively linked to a heterologous and/or endogenous gene. In addition, the invention relates to the use of these constructs in the form of expression vectors, recombinant vectors and in plants, plant cells or transgenic protoplasts. The invention further describes a method of obtaining transgenic plants, plant cells or protoplasts using said constructs containing the promoter and the regulatory regions of the invention. Thus, the expression of the transgene only in the part of interest enables the accumulation of the exogenous transcript only in the root, favoring the implementation of strategies that aim at the increase in added value, the generation of cultivars that are more adapted to environmental stress, the pathogens and pests, agrochemicals, besides plants with a high nutritional value and high therapeutic value. In addition to these advantages, the present invention is a new alternative for the expression systems in plant organisms and can be used for the generation of new cultivars and improvement programs. The invention has the objective of increasing economical, social and environmental benefits, as well as biosecurity, associated to genetic transformation.

### BACKGROUND OF THE INVENTION

Manioc (Manihot esculenta Crantz) belongs to the Euphorbiaceae family, is native of South America, and is one of the most important tropical food crops for over 600 million people around the world. Basically, each part of the plant can be used, but the roots are the most widely used product. In developing countries, manioc roots are often the only source

of calories.

Stored roots develop from primary roots by cell division and differentiation of parenchyma cells of the secondary xylem (Rateaver B (1951). Anatomy and Regeneration in the Stem and Root of Manihot utilissima Pohl. Doctoral thesis, University of Michigan, Ann Arbor, 1-100; Castilloa J J, Castilloa A and Pino L T (1997). Notas sobre histología foliar y radical en yucca (Manihot esculenta Crantz). In: La Yuca Frente al Hambre del Mundo Tropical, Universidad Central de Venezuela, Caracas, 77-100). An anatomical model with three systems of compartmentalization of tissues has been used in studies of gene expression (De Souza C R, Carvalho L J, De Almeida E R and Gander E S (2002). Towards the identification of cassava root protein genes. Plant Foods Hum. Nutr. 57; 353-363; De Souza C R, Carvalho L J, De Almeida E R and Gander E S (2006). A cDNA sequence coding for a glutamic acid-rich protein is differentially expressed in cassava storage roots. Protein Pept. Lett. 13:653-657). According to this model, the tissue system I is composed by phellogen and phelloderm, tissue of system II of the phloem and vascular cambium, tissue and system III of the secondary xylem with its parenchyma highly specialized cells rich in starch.

Pt2L4 is an alcohol-soluble protein predominantly expressed in the tissue of system III, which contains xylem and parenchyma cells, with starch granules (De Souza C R, Carvalho L J, De Almeida E R and Gander E S (2002). Towards the identification of cassava root protein genes. Plant Foods Hum. Nutr. 57:353-363). The amino acid composition of protein Pt2L4 revealed that the most abundant amino acids are glutamic acid (31.6%), alanine (16.94%), valine (13.55%) and proline (11.29%) (De Souza C R, Carvalho L J, De Almeida E R and Gander E S (2006). Proteins Pt2L4 and C54 are 60% identical with similar molecular weights (16.7 and 18.0 kDa, respectively) and isoelectric points (3.70 and 3.97) (Zhang P, Bohl-Zenger S, Puonti-Kaerlas J, Potrykus I, et al. (2003). Two cassava promoters related to vascular expression and storage root formation. Planta 218: 192-203). There are two or more homologous genes coding for proteins rich in glutamic acid in the manioc genome according to Southern blot analysis (Zahng P, Bohl-Zenger S, Puonti-Kaerlas J, Potrykus I, et al (2003). Two cassava promoters related to vascular expression and storage root formation. Plant 218:192-2003; De Souza C R, CArvalho L J, De Almeida E R and Gander E S (2006). A cDNA sequence coding for a glutamic acid-rich protein is differentially expressed in cassava storage roots. Protein Pept. 13: 653-657). Studies have disclosed that their transcripts are more strongly expressed in vascular tissues and in parenchyma cells of storage roots, indicating an important role in the formation of storage roots (Zahng P, Bohl-Zenger S, Puonti-Kaerlas J, Potrykus I, et al (12003). Two cassava promoters related to vascular expression and storage root formation. Planta 218: 192-203; De Souza C R, Carvalho L J and De Mattos Cascardo J C (2004). Comparative gene expression study to identify genes possibly related to storage root formation in cassava. Proteion Pept. Lttt. 11: 577-582; De Souza C R, Carvalho L J, De Almeida E R and Gander E S (2006). A cDNA sequence coding for a glutamic acid-rich protein is differentially expressed in cassava storage roots. Protein Pept. Lett. 13: 653-657). Besides, Zhang et al (Zanch P, Bohl-Zenger S, Puonti-Kaerlas J, Potrykus I, et al (2003). Two cassava promoters related to vascular expression and storage root formation. Planta 218:192-203) report greater activity of promoter C54 in the vascular cambium and parenchyma cells rich in starch of transgenic tubercle roots of cassava containing this promoter, fused to the β-glucuronidase reporter gene (GUS).

In spite of the recent advances in isolating and characterizing endogenous promoters from manioc, only a few tissue and organ-specific promoters were identified. The identification of tissue-specific promoters is essential for the manioc genetic engineering, which has been used to raise the nutritional value of the roots, as well as to produce plants with greater resistance to viral diseases and insects, pests and with reduced cyanogenic contents (Tayler N, Chavarriaga P, Reamakers K, Siritunga D, et al (2004). Development and application of transgenic technologies in cassava. Plant Mol. Biol. 56: 671-688).

Literature data indicates evolution in the application of genetic transformation with the appearance of various generations of transgenics. The first and second generation of transgenic plants used constructive promoters like the cauliflower mosaic virus 35S promoter (CaMV 35S) (Odell, J. T., Nagy, F. & Chua, N-H. 1985 Identification of DNA sequences required for the activity of the cauliflower mosaic virus 35S promoter. Nature, v. 313, p. 810-812), gene promoters found in the T-DNA of Agrobacterium tumefaciens, for example, the nopaline syntase enzyme gene promoter (Bevan, M. W., Barnes, W. M. & Chilton, M. D. 1983 Structure and transcription of the nopaline syntase gene region of T-DNA. Nucleic Acids Research, v. 11, n. 2, p 369-385) and promoters of genes coding for proteins that are highly preserved and involved in vital processes of virtually all organisms like ubiquitin (Toki S., Takamatsu S., Nojiri C., Ooba S., Anzai H., Iwata M., Christensen A. H., Quail P. H. & Uchimiya H. 1992 Expression of a Maize Ubiquitin Gene Promoter-bar Chimeric Gene in Transgenic Rice Plants. Plant Physiol. November; 100(3)?1503-7) and actin (An Y. Q., McDowell J. M., Huang S., McKinney E. C., Chambliss S. & Meagher R. B. 1996 Srong, constitutive expression of the Arabidopsis ACT2/ACT8 actin subclass in vegetative tissues. Plant J. July; 10(1):107-21). However, the third generation of transgenic plants is characterized, among other features, by the use of tissue-specific promoters like: epidermal cell specific promoters (Hooker T. S., Millar A. A. & Kunst L. 2002. Significance of the expression of the CER6 condensing enzyme for culticular wax production in Arabidopsis. Plant Physiol. August; 129(4): 1568-80), from phloem (Okumoto S., Koch W., Tegeder M., Fischer W. N., Biehl, A., Leister D., Stierhof Y. D. & Frommer W. B. 2004. Root phloem-specific expression of the plasma membrane amino acid proton co-transporter AAP3. J. Exp Bot. October; 55(406):2155-68), from pollen (Wakeley P R, Rogers, H J, Rozycka M, Greenland A J, Hussey P J. A maize pectin methylesteraze-like gene, ZmC5, specifically expressed in pollen. Plant Mol Biol. 1998 May; 37(1):187-92) or biotic stress induced (Himmelbach A., Liu L., Zierold U., Altschmied L., Maucher H., Beier F., Müller D., Hensel G., Heise A., Schützendübel A., Kumlehn J. & Schweizer P. 2010. Promoters of the barley germin-like GER4 gene cluster enable strong transgene expression in response to pathogen attack. Plant Cell. 2010. March; 22(3)937-52. Epub Mara 19), abiotics (Rai M., He C. & Wu R. 2009. Comparative functional analysis of three abiotic stress-inducible promoters in transgenic rice. Transgenic Res. October; 18(5): 787-99) and chemical (Tomsett, A. Tregova, A. Garoosi and M. Caddick, Ethanol-inducible gene expression: first step towards a new geeen revolution?, Trends Plant Sci. 9 (2004), pp. 159-161) to promote a punctual transgene expression and only when necessary.

Obtaining and making available promoters capable of limiting gene expression in time and space may be one of the ways to balance the benefits of transgenics and its restrictions.

Promoter is a set of transcription control modules, organized around the initiation site of the RNA polymerase II enzyme (Potenza, C.; Aleman, L. & Sengupta-Gopalan, C. 2004. Targeting transgene expression in research, agricultural and environmental applications: Promoters used in plant transformation. In Vitro Cellular Development Biological Plant V. 40, p. 1022), which contain specific sequences recognized by proteins involved in the transcription. Different classes of promoters have been described in the literature, based on their expression profile. Among them is that of constitutive promoters, which are active in all tissues and at every phase of development of the organism, such as for instance, CaMV35S (Odell, J. T., Nagy, F. & Chua, N-H. 1985. Identification of DNA sequences required for the activity of the cauliflower mosaic virus 35S promoter. Nature. V. 313, p. 810-812). On the other hand, the tissue/organ-specific promoters promote the expression of its related gene only in its target-tissue/organ like fruit (Aktinson R. G., Bolitho K. M. Wright M. A., Iturriagagoitia-Bueno T., Reid S. J. & Ross G. S. 1998 Apple ACC-oxidase and polygalacturonase: ripening-specific gene expression and promoter analysis in transgenic tomato. Plant Mol Biol. Cot; 38(3):449-60), grain seed (Paine J. A., Shipton C. A., Chaggar S., Howlls R. M. J., Vernon G., Wright S. Y., Hinchiliffe E., Adams J. L., Silverstone A. L. & Drake R. 2005. Improving the nutritional value of Golden Rice through increased pro-vitamin A content. Nat. Biotechnol. April; 23(4):482-7), root/tubercles (Visser R. G., Stolte A. & Jacobsen E. 1991. Expression of a chimaeric granule-bound starch synthase-GUS gene in transgenic potato plants. Plant Mol Bio. October; 17(4):691-9), flowers (Annadana S., Beekwilder M. J., Kuipers G., Visser P. B., Outchkourov N. Pereira A., Udayakumar M., De Jong J. & Jongsman M. A. 2002. Clonign of the chrysanthemum UEP1 promoter and comparative expression in florets and leaves of Dendranthema grandiflora. Transgenic Res. August; 11(4):437-45) and leaves (Nomura M., Katayama K., Nishimura A., Ishida Y., Ohta S., Komari T., Miyao-Tokutomi M., Tajima S. & Matsuoka M. 2000. The promoter of rbcS in a C3 plant (rice) directs organ-specific, light-dependent expression in a C4 plant (maize), but does not confer bundle sheath cell-specific expression. Plant Mol Biol. September; 44(1):99-106). There are still the responsive or inducible promoters, which are activated by a determined situation, in response to biotic, abiotic or chemical stresses. Promoters isolated from a given organism may be used to regulate a gene of another organism by means of chimeric gene constructs.

There are numberless tissue-specific promoters described for plants, as is the case of the seed-specific expression (WO8903887), tubercle (as mentioned in patent application US20030175783, Keil et al., 1989 (EMBO J. 8: 1323:12330), leaves (as mentioned in patent application US20030175783, Hudspeth et al., 1989 Plant Mol Biol 12:579-589), fruit (Edwards and Coruzzi (1990) Annu. Rev. Genet. 24, 275-303 and U.S. Pat. No. 5,753,475), stem (as mentioned in patent application US2003017583, Keller et al., 1988 EMBO J. 7:3625-3633), vascular tissues (as mentioned in patent application US2003017583, Peleman et al., 1989 Gene 84:359-369 and Schmülling et al. (1989) Plant Cell 1, 665-670), root (US20060143735 and as mentioned in patent application US20030175783, Keller et al., 1989 Genes Devel. 3:1639-1646), stamens (WO8910396, WO9213956), specific promoters of the dehiscence zone (WO9713865) and meristem (Ito et al (1994) Plant Molecular Biology, 24, 863-878).

In the present invention, we present a promoter isolated from manioc (Manihot esculenta Crantz), specific for the root, generating the possibility of expressing a protein of interest specifically in the root. At present, the promoters used in obtaining commercial transgenics are often constitutive and promote the expression of the transgene in all the tissues of the plant and in all the phases of development. This characteristic causes energetic wear in the plant, since there is metabolic waste in the production of a protein in amounts and at sites where it is unnecessary and may lead to decrease in productivity. Besides, it is desirable, from the commercial point of view, to aggregate value to economically important crops, as is the case of golden rice, which is rich in β-caroten (Beyer P. Golden Rice and ‘Golden’ crops for human nutrition. 2010. May 15. N Biotechnol. [Epub ahead of print]. Http://www.sciencedirect.com/science? Ob=ArticleURL& udi=B8JG4-5033Y3B-2& user=7430124& coverDate=05%2F15%2F2010& rdoc=1& fmt=high&0-Rig=search& sort=d& docanchor=&view=c& acct=C000012878& version=1& urlVersion=0& userid=7430124&md5=a9c6d210db86be4e6946f0f1f69766c0). Another important aspect is the one makes the biosafety tests easier, since the expression of the gene is restricted to a single organ.

The more and more frequent use of transgenics as a means to solve problems of economically important crops occurs because transgenics enables one to incorporate desirable characteristics in a direct matter, independent of barriers between species.

Most transgenic plants launched on the market until now use constitutive promoters, which activate the expression of their transgene in every the tissue of the plant. Another limiting factor is the technological dependence, since the uses of the promoters available at present are protected by patents. The invention proposed herein can further be viewed as an alternative of existing promoting regions, chiefly in cultivars and improvement programs involving a cultivar of the genus Manihot, since the promoter sequence proposed was isolated from it, and so it is not considered a proper transgenic sequence.

The present-day agronomical scene is subject to impacts caused by weather changes, which cause serious unbalance in the environment and in agriculture, besides the increase in population, which is a factor that generates environmental unbalance due to the demand for space to increase the agricultural production. With a view to reduce these impacts, it is necessary to handle the natural resources like water and space in a sustainable manner, as well as adversities like drought, pests and pathogens. Thus, it is necessary to develop plants that are more suitable for this scene, and transgenics is a tool that accelerates the obtainment of these cultivars.

Until now, only two promoter sequences from manioc root were isolated and are in the process of patenting ion Germany (European Patent Office, 80298 Munich, Germany). These sequences are promoters of genes C15 and C54, which encode proteins Cytochrom p450 and C54, respectively.

In view of the shortage of available promoters for genetic improvement of manioc, the DNA sequence (SEQ ID NO1) containing the Mec1 gene promoting region, which encodes protein Pt2L4, rich in glutamic acid with differential expression in the root of the manioc reserve has been isolated and characterized.

In the face of the above explanation, the present invention refers to a new promoter sequence and root-specific regulatory regions for the expression of a gene of interest only in this region. Besides presenting advantages like the directed expression only in the root and being used in Manihot cultivars and in programs for the improvement of the same genus, the invention further brings a new alternative to the expression systems in plants.

### SUMMARY OF THE INVENTION

The invention refers to a new promoter and regulatory regions for specific expression of the gene in plant root. In this way, the expression of the transgene only in the part of interest enables accumulation of exogenous transcript only in the root, favoring the implementation of strategies that aim at the increase of the aggregated value, the generation of cultivars that are more adapted to the environmental stress, pathogens and pests, agricultural chemicals, in addition to vegetable organism having high nutritional value and high therapeutic value. In addition to these advantages, the present invention is a new alternative to the systems of expression in vegetable organisms and can be used for generating new cultivars and improvement programs. The invention has the objective of increasing the economical, social and environmental benefits, as well as biosafety, associated to genetic transformation.

In a first embodiment, the present invention provides a polynucleotide sequence that is substantially similar to the SEQ ID NO1; reverse sequence of SEQ ID NO1; probes and primers corresponding to the SEQ ID NO1.

In another aspect, the present invention provides chimeric genes comprising polynucleotide of the present invention, either alone or in combination with one or more known polynucleotides, together with cells and organisms comprising these chimeric genes.

In a related aspect, the present invention provides recombinant vectors comprising, in direction 5′-3′, a sequence of promoters and/or polynucleotide regulatory regions of the present invention, a polynucleotide to be transcribed and a gene determination sequence. The polynucleotide to be transcribed may comprise an open reading frame of a polynucleotide encoding a polypeptide of interest, or may be a non-coding region, or non-translated region, of a polynucleotide of interest. The open reading matrix may be oriented in a “sense” or “antisense” direction. Preferably, the gene determination sequence if functional in a host plant. More preferably, the gene determination sequence is that of the gene of interest, but it may be others described in the prior art (see Benjamin Lewin, Genes VIII, chapter 9) such as the A. tumefasciens nopaline synthase terminator. The recombinant vectors may further include a marker for identification of transformed cells.

In another aspect, cells of transgenic plants comprising the recombinant vector of the present invention are provided, together with organisms like plants comprising these transgenic cells, and fruits, seeds and other products, derivatives or progeny of these plants. The propagule of inventive transgenic plants are included in the present invention.

In another aspect of the present invention, a method is provided for modifying the expression of genes in an organism such as a plant, including the stable incorporation into the genome of the organism containing the recombinant vector of the present invention.

In another aspect of the present invention, a method is provided for producing a transformed organism such as a plant having the modified expression of a polypeptide. This method comprises transforming a plant cell with the recombinant vector of the present invention to provide a transgenic cell under conditions that lead to the generation and growth of the mature plant.

In a further aspect of the present invention, a method is provided for identifying a gene responsible for a desired function or phenotype. The method comprises: 1) transforming a plant cell containing a recombinant vector comprising a promoter sequence and/or polynucleotide regulatory regions of the present invention, operatively attached to a polynucleotide to be tested; 2) cultivating the plant cell under conditions that lead to regeneration and growth of the mature plant, so as to provide a transgenic plant; 3) comparing the phenotype of the transgenic plant with the phenotype of non-transformed plants or of a wild type.

The above-mentioned and additional aspects of the present invention and the way to obtain them will be apparent, and the invention will be better understood by reference to the “Detailed Description of the Invention”.

### BRIEF DESCRIPTION OF THE FIGURES

FIG. 1 shows levels of transcription of Mec1 in the storage root and tissues. O total RNA (10 μg) of: L1-L5 five layers of root storage tissue; (Ct.): cotyledons; (YS): young radical; (SP): bark trunk; (Pt.): petiole and (Lf.): leaves was separated in agaroseformaldehyde gel, transferred to a Hybond-N+membrane and probed with cDNA Mec1 marked with [32P] dCTP. Hybridization with ribosomal RNA 28S is included as standard;

FIG. 2 shows Genic construct pMec1-GUS;

FIG. 3 shows Detection of the activity of GUS in embryo axis of beans bombarded with pCAMBIA-Mec1 (b, e, f) pCAMBIA 3201 (c) and pCAMBIA 3201 with the promoter 35S deleted. Bars=0.5 mm.

### DETAILED DESCRIPTION OF THE INVENTION

The present invention refers to a promoter and root-specific regulatory regions for the expression of gene of interest only in this region of the transgenic vegetable organism.

In the description hereinafter, a number of terms is used extensively. The following definitions are provided in order to facilitate the understanding of the invention.

A “chimeric gene” is a gene comprising a promoter and a coding region of different origins. In the case of the present invention, the chimeric gene comprises the polynucleotide of the present invention attached to regions encoding endogenous and/or exogenous genes.

A “consensus sequence” is an artificial sequence in which the base at each position represents the base that is most often found in the present sequence, in comparing different alleles, genes or organisms.

A “promoter” is that portion of the DNA upstream of the encoding region, which contains binding sites for RNA polymerase II to start the DNA transcription.

“Expression” is the transcription or translation of a structural gene, either endogenous or heterologous.

“GC box” is a common element on the promoter that can enhance the activity of the promoter.

“TATA box” is an element in the promoter, located approximately 30 bases upstream of the transcription starting site. The TATA box is associated with transcription factors in general, including RNA polymerase II.

The term “gene” means a physical and functional unit of heredity, represented by a DNA segment that encodes a functional protein or RNA molecule.

An “endogenous gene” is a gene belonging to the cell or to the organism.

A “heterologous gene” is a gene isolated from a donor organism and recombined in the transformed receptor organism. It is a gene that does not belong to the cell or to the organism.

A “reporter gene” is an encoding unit the product of which is easily tested, for instance, genes CAT, GUS, GAL, LUC and GFP. The expression of a reporter gene may be used for testing the function of a promoter bound to this reporter gene.

The term “propagule” as used in the present invention means any part of a plant that can be sued in sexual or asexual reproduction or propagation, including the seedlings.

“Sense” means that the polynucleotide sequence is in the same orientation 5′-3′ with respect to the promoter.

“Antisense” means that the polynucleotide sequence is in the contrary orientation with respect to the orientation 5′-3′ of the promoter.

As used herein, the term “x-mer”, as a reference to a “x”-specific value, refers to a sequence comprising at least one specific number (“x”) of residues of the polynucleotide identified as SEQ ID NO1. According to preferred embodiments, the value of x is preferably at least 20, more preferably at least 40, still more preferably at least 60 and more preferably at least 80. Thus, polynucleotides of the present invention comprise a polynucleotide of 20 mers, 40 mers, 60 mers, 80 mers 100 mers, 120 mers, 150 mers, 180 mers, 220 mers, 250 mers, 300 mers, 400 mers, 500 mers or 600 mers identified as SEQ ID NO1 and variants thereof.

The term “polynucleotide(s)” as used herein means single-strand or double-strand polymer of deoxyribonucleic or ribonucleic bases and includes corresponding RNA and DNA molecules, including HnRNA and mRNA molecules having both “sense” and “antisense” strands, and comprises cDNA, genomic DNA, and recombinant DNA, as well as completely or partially synthesized polynucleotides. A HnRNA molecule contains introns and corresponds to a DNA molecule in a generally one-to-one manner. An RNAm molecule corresponds to a DNA and HnRNA molecule, from which the introns have been excised. A polynucleotide can consist of a complete gene or any portion thereof. The operable “antisense” polynucleotides can comprise a fragment of the corresponding polynucleotide, the definition of “polynucleotide” thus including all these operable antisense fragments. The antisense polynucleotides and techniques involving antisense polynucleotides are well known from the prior art (Sambrook, J.; E. F. Fritsch and T. Maniatis—Molecular cloning. A laboratory manual, 2^{nd} ed., Cold Spring Harbor Laboratory Press, 1989).

The polynucleotides described in the present invention are preferably about 80% pure, more preferably at least about 90% pure, and more preferably at least about 99% pure.

The term “oligonucleotide” refers to a relatively short segment of a polynucleotide sequence, generally comprising from 6 to 60 nucleotides. These oligonucleotides may be used as probes or primers, wherein the probes may be used for use in hybridization tests and primers for use in DNA amplification by polymerase chain reaction.

The term “probe” used in the present invention refers to an oligonucleotide, polynucleotide or nucleic acid, being RNA or DNA, if occurring naturally as in a digestion of purified or synthetically produced restriction enzyme, which is capable of annealing with or specifically hybridizing with a nucleic acid containing probe complementary sequences. A probe may further be of single- or dual chain. The exact length of the probe will depend on many factors, including temperature, origin of the probe and use of the method. For instance, depending on the complexity of the target sequence, the oligonucleotide probe typically contains 15-25 or more nucleotides, although it may contain less nucleotides. The probes herein are selected to be complementary to differentiate chains of a sequence from a particular nucleic acid. This means that the probe may be sufficiently complementary to be capable of “hybridizing specifically” or annealing with its respective target chains under a number of pre-determined conditions. As a result, the probe sequence does not need to reflect exactly the complementary sequence of the target. For example, a complementary nucleotide fragment may be attached to the end 5′ or 3′ of the probe, with the rest of the probe sequence being complementary to the target chain. Alternatively, non-complementary bases or long sequences may be intercalated within the probe if it has sufficient complementariness with the sequence of the target nucleic acid to anneal specifically with it.

The term “primer” as used herein refers to an oligonucleotide, being RNA or DNA, single- or double chain, derived from a biologic system, generated through digestion with restriction enzymes, or synthetically produced, which, when placed in a suitable environment, is capable of acting functionally as a starter of a synthesis of a mold-dependent nucleic acid. When presented with an appropriate nucleic acid mold, adequate nucleoside triphosphates, nucleic acid precursors, a polymerase enzyme, suitable co-factors and conditions such as adequate temperature and pH, the primer may be extended at its 3′ terminal by adding nucleotides through the action of a polymerase or with similar activity to produce a first extension of the product. The primer may vary in length, depending on particular conditions and requirements for application. For instance, in diagnostic applications, the oligonucleotide primer typically has from 15 to 25 or more nucleotides in length. The primer should have sufficient complementariness with the desired mold to start the synthesis of the extension of the desired product. This does not mean that the sequence of the primer should represent an exact complement of the desired mold. For instance, a non-complementary nucleotide sequence may be attached to the 5′ end of a complementary primer. Alternatively, non-complementary bases may be intercalated within the oligonucleotide sequence of the primer, as long as the primer has sufficient complementariness with the sequence of the desired mold chain to promote functionally a mold-primer complex for synthesis of the product extension.

The term “specifically hybridizing” refers to the association between two single-chain nucleic acid molecules that have sufficiently complementary sequences to enable such hybridization under pre-determined conditions generally described in the prior art (apostila: Tecnologia de DNA recombinante. Universidade de São Paulo, Capítulo 1, 2003).

In particular, the term refers to hybridization of an oligonucleotide with a substantially complementary sequence containing a single-chain DNA or RNA molecule of the present invention. Appropriate conditions required for carrying out specific hybridization in single-chain nucleotide acid molecules of varying complementariness are well described in the prior art (apostila: apostila: Tecnologia de DNA recombinante. Universidade de Sao Paulo, Capítulo 4, 2003). A common formula to calculate the stringency conditions required for hybridization between nucleic acid molecule follow below (Sambrook et al, Molecular Cloning, A Laboratory Manual, 2^{nd} ed (1989), Cold Spring Harbor Laboratory Press): Tm=81.5° C.+16.6 Log Na++0.41(% G+C)−0.63(% formamide)−600/PB in duplex(probe).

As illustrated by the above formula, using Na+=0.368 and 50% formamide, with contents of GC of 42% and an average probe size of 200 bases, the Tm will be 57° C.

Probes or primers are described as corresponding to the polynucleotide of the present invention, identified as SEA ID NO1 or a variant thereof, if the oligonucleotide probe or primer, or its complement, is contained in the specific sequence as SEQ ID NO1, or a variant of the latter.

The term “oligonucleotide” refers to primers and probes of the present invention, and is defined as being a nucleotide acid molecule comprising two or more ribo- or deoxyribonucleotides, preferably more than three. The exact size of the oligonucleotides depends on various factors and on the particular application and use of the oligonucleotides. Preferred oligonucleotides comprise 15-50 consecutive pairs of base complementary to SEQ ID NO1. The probes may be easily selected by using procedures that are well described in the prior art (Sambrook et al “Molecular Cloning, a laboratory manual, CSHL Press, Cold spring Harbor, N.Y., 1989), taking into consideration DNA-DNA hybridization stringencies, recombination and fusion temperatures, and potential for the formation of bonds and other factors, which are known from the prior art.

The definition of the terms “complement”, “reversed complement” and “reverses sequence”, as used herein, is illustrated by the following example. For the sequence 5′AGTGAAGT3′, the complement is 3′TCACTTCA5′, the reversed complement is 3′ACTTCACT5′ and the reversed sequence is 5′TGAAGTGA3′.

As used herein, the term “variant” or “substantially similar” comprise amino acid or nucleotide sequences different from specifically identified sequences, in which one or more nucleotides or residues of amino acids is deleted, substituted or added. The variants may be allelic variants, naturally occurring variants, or non-naturally occurring variants. The varying or substantially similar sequences refer to fragments of nucleic acids that may be characterized by the similarity percent of the nucleotide sequences with the nucleotide sequences described herein (SEQ ID NO1), as determined by common algorithms employed in the prior art. The preferred nucleic acid fragments are those of which the nucleotide sequences have at least about 40 or 45% sequence identity, preferably about 50% or 55% sequence identity, more preferably about 70% or 75% sequence identity, more preferably about 80% or 85% sequence identity, still more preferably about 90%, 91%, 92%, 93%, 94%, 95%, 96%, 97%, 98% or 99% sequence identity as compared with the reference sequence. The percent identity is determined by aligning two sequences to be compared, determining the number of identical residues in the aligned portion, dividing this number by the total number of residues in the researched sequence, and multiplying the result by 100. This alignment may be made through software existing on the Internet, one of which is the BLASTN, which is available from the site of the National Center for Biotechnology Information/NCBI (www.ncbi.nlm.nih.gov).

The term “vector” refers to a replicon, such as plasmide, cosmide, bacmide, phage or virus, in which other genetic sequences or elements (be it DNA or RNA) can be linked to be replicated together with the vector. Preferably the vector derived from a virus is selected from bacteriophages, vaccines, retrovirus or virus of bovine papiloma. The “recombinant vector” results from the combination of a commercial vector with chimeric genes, or the polynucleotide of the present invention operationally linked to an endogenous and/or heterologous polynucleotide of interest, which in turn is operatively linked to a termination signal. Such vectors can be obtained commercially, including from Clontech Laboratories, Inc (Palo Alto, Calif.) Stratagene (La Jolla, Calif.), Invitrogen (Carlsbad, Calif.), New England Biolabs (Beverly, Mass.) and Promega (Madison, Wis.). A few examples of vectors that may be used in the present invention, but not limited thereto, are the vectors pGEM-T, pGEMTeasy, pCAMBIA 3201.

The term “expression enhancing sequence”, is known as enhancers, which may be very far from the promoter (either upstream or downstream) and which potentiates the transcription rate. These enhancers are not specific and potentiate the transcription of any promoter that is in their vicinity. The efficiency of the expression of a gene in a specific tissue depends on the adequate combination and integration of the enhancers, of the promoters and of the adjacent sequences.

The first enhancer discovered to stimulate the transcription of eukaryotic genes was the SV40 (present in the genome of the Simian virus 40) After the discovery of the SV40 enhancer, hundreds of other enhancers like HSV-1, AMV, HPV-16 were identified, in other viral genomes in the DNA of eukaryotic cells (Lodish et al, Biologia cellular e molecular, 4^{th} ed., p. 368).

The term “operatively linked” means that the regulatory sequences necessary to express the coding sequence are placed in the DNA molecule at appropriate positions relative to the coding sequence for expressing the coding sequence. The same definition is applied sometimes for arranging coding sequences and transcription controlling elements (for example, promoters, auxiliaries or enhancers and termination elements or sequences) in the expression vector. An exogenous coding sequence is typically flanked by operatively linked regulatory regions that regulate the expression of the exogenous coding region in a transformed cell (which may be a vegetable or animal microorganism). A typical regulatory region operatively linked to an exogenous coding region includes a promoter, that is, a nucleic acid fragment that can cause transcription of exogenous coding regions, positioned at region 5′ of the exogenous coding region. In the case of the present invention, the regulatory region refers to the regions substantially similar to the SEQ ID NO1. In order to aid in enhancing the transcription of a determined polynucleotide, the promoter sequence of the present invention may be linked to other regulatory sequences already described, such as: ATATT (an element of strong expression in the root), AACAAAC and GCCACCTCAT (SEQ ID NO: 2) (elements referring to the seed-specific expression), CACGTG and CCTACC (both sequences may be stimulated to a stress factor), among others. Ai-Min Wu et al, Isolation of a cotton reversibly glycosylated polypeptide (GhRGP1) promoter and its expression activity in transgenic tobacco, Journal of Plant Physiology 163 (2006) 426-435).

A “termination sequence” is a DNA sequence that signals the transcription end. Examples of termination sequences, not limited thereto, SV40 termination signal, HSV TK termination signal, termination signal of the Agrobacterium tumefasciens nopaline synthase gene (NOS), termination signal of the octopine synthase gene, termination signal of the 19S and 35S CaMV gene, termination signal of the maize alcohol dehydrogense gene, termination signal of the manopine synthase gene, termination signal of the beta-phaseolin gene, termination signal of the ssRUBISCO gene, termination signal of the sucrose synthase gene, termination signal of the virus that attacks Trifolium subterranean (SCSV), termination signal of the Aspergillus nidulans trpC gene and other similar ones. The present invention provides an isolated polynucleotide regulating region that may be employed in manipulating plant phenotypes, together with isolated polynucleotides comprising these regulatory regions. More specifically, the present invention relates to the promoter or regulatory sequence that occurs naturally in manioc plants (Mahihot esculenta Crantz), responsible for the expression of the Mec1 gene in roots of this plant species. The manioc promoter and regulatory regions were isolated from the Mec1 gene which is responsible for the expression of the Pt1L4 protein in manioc roots and were called in the present invention Mec1 (SEQ ID NO1).

The amount of a specific polypeptide of interest can be increased or reduced by incorporating additional copies of genes, or encoding sequences, coding the polypeptide, operatively linked to the promoter sequence of the present invention (SEQ ID NO1), in the genome or an organism such as a plant. Similarly, an increase or decrease in the amount of the polypeptide can be obtained by transformation of a plant with antisense copies of these genes.

The polynucleotide of the present invention has been isolated from manioc plants, more specifically of Manihot esculenta Crantz, but it may be alternatively synthesized by using conventional synthesis techniques. Specifically, the isolated polynucleotide of the present invention includes the sequence identified as SEQ ID NO1; the reverse complement of the sequence identified as SEQ ID NO1; reverse complement of the sequence identified as SEQ ID NO1.

The polynucleotide of the present invention may be identified in genomic DNA sequences of plants for which the information of the genome sequence is available to the public, or isolated from polynucleotide libraries, or may be synthesized by using techniques that are well known from the prior art (Sambrook et al “Molecular Cloning, a laboratory manual”, CSHL Press, Cold Spring Harbor, N.Y., 1989). The polynucleotide may be synthesized, for instance, by using automated synthesizers of oligonucleotides (for example, synthesizer of DNA OLIGO 1000M Backman), to obtain polynucleotide segments of up to 50 or more nucleic acids. A plurality of these polynucleotides may then be linked by using standard DNA manipulation techniques that are well known from the prior art (Sambrook et al “Molecular Cloning, a laboratory manual”, CSHL Press, Cold Spring Harbor, N.Y., 1989). A conventional and exemplary polynucleotide synthesis technique involves synthesizing a single-strand polynucleotide segment having, for instance, 80 nucleic acids, and hybridizing this segment to a complementary 85-nucleic acid segment, synthesized to produce a 5-nucleotide ‘overhang”. The next segment may then by synthesized in a similar way, as a 5-nucleotide overhang in the opposite strand. The “sticky” or cohesive ends ensure an adequate linking when the two portions are hybridized. Thus, the polynucleotide of the present invention may be synthesized completely in vitro.

As can be seen from the above, the promoter sequence of the present invention may be employed in recombinant and/or expression vectors to activate the transcription and/or expression of a polynucleotide of interest. The polynucleotide of interest may be either endogenous or heterologous to an organism, for instance, a plant, to be transformed. The recombinant and/or expression vectors of the present invention may thus be employed to modulate transcription and/or expression levels of a polynucleotide, for instance, a gene that is present in the wild-type plant, or may be employed to promote transcription and/or expression of a DNA sequence that is not found in the wild-type plant, including, for instance, a gene encoding a reporter gene, such as GUS.

In some embodiments of the present invention, the polynucleotide of interest comprises an open reading matrix encoding a polypeptide of interest. The open reading frame is inserted into the vector in a sense orientation and the transformation with this genetic construct/recombinant vector will generally result in super-expression of the polypeptide selected. The polypeptide of interest, which will be regulated by the promoter of the present invention, may be inserted into the vector in the sense, antisense orientation of in both directions. The transformation with a recombinant and/or expression vector containing the promoter of the invention regulating the expression of the polynucleotide of interest in the antisense orientation or in both orientations (sense and antisense) will generally result in the reduced expression of the polypeptide selected.

The polynucleotide of interest, as a coding sequence, is linked to in an operative manner in a sequence of the polynucleotide promoter of the present invention, so that the host cell is capable of transcribing an RNA activated by the promoter sequence linked to the polynucleotide of interest. The polynucleotide promoter sequence is generally positioned at the end 5′ of the polynucleotide to be transcribed. The use of a tissue-specific promoter, like a sequence of the nucleotide of manioc (Manihot esculenta Crantz), responsible for the expression of the Mec1 gene identified as SEQ ID NO1, will affect the transcription of the polynucleotide of interest only in the endosperm of the transformed plant.

The recombinant vector or expression vector of the present invention may also contain a selection marker that is effective in cells of the organism such as a plant, to enable detection of transformed cells containing the inventive recombinant vector. These markers, which are well known, typically impart resistance to one or more toxins. An example of this marker is the gene nptII, the expression of which results in resistance to canamicyn or neomicyn antibiotics that are generally toxic to plant cells in a moderate concentration. The transformed cells may thus be identified by their capability to grow in a medium containing the antibiotic in question. Other markers that may be used for constructing recombinant and/or expression vectors containing the polynucleotide of the present invention may be, but are not limited thereto: hpt gene imparts resistance to the antibiotic hygromicyn, manA gene and bar gene.

The system that uses the gene manA (which encodes enzyme PMI-phosphomannose isomerase) of Escherichia coli (Miles and Guest, 1984. Complete nucleotide sequence of the fumarase gene fumA, of E. coli. Nucleic Acids Res. 1084 April 25; 12(8): 3631-3642), having mannose as selective agent, is one of the new systems suggested as alternative to the first two described above (Joersbo et al, 1998 Parenmeters iteracting with mannose selection employed for the production of transgenic sugar beet, Physiologia Plantarum Volume 105 Issue 1 Page 109—January 1999, doi:10.1034/j.1399-3054.1999.105117.x). The plant species that do not metabolize mannose undergo a severe inhibition of growth when it is offered as the only source of carbon in a culture medium. The adverse and inhibitory effects of the use of mannose are consequences of the accumulation of mannose-6-phosphate, a product of mannose phosphorylation by a hexokinase. PMI promotes interconversion of mannose-6-phosphate and fructose-6-phosphate, thus enabling the former to be catabolized in the glycolytic pathway (Ferguson and Street, 1958. Analise de sistemas gene marcador/agente seletivo alternativas para seleção positiva de embriões somáticos transgênicos de mamoeiro (Analysis of alternative marker gene/selective gene for positive selection of transgenic somatic embryos of papaya plant) Ver. Bras. Fisiol. Veg., 2001, vol. 13, no. 3, p. 365-372. ISSN 0103-3131.: Malc E T al, 1967 Advances ion the selection of transgenic plants using non-antibiotic marker genes, Physiologia Plantarum Volume 111 Issue 3, Page 269 —March 2001 doi:10.1034/j.1399-3054.2001.1110301.x). The gene bar (which encodes the enzyme PAT=phosphinothricin-N-acetyltransferase) of Streptomyces hygroscopicus (Murakani et al, 1986 The bialaphos biosynthetic genes of Streptomyces hygroscopicus; molecular cloning and characterization of the gene cluster. Molecular and General Genetics, 205: 42-50, 1986), having ammonium glyphosinate (PPT) as selective agent, is, among the systems of the herbicide tolerant gene type, one of the most widely employed by genetic engineering in the development of vegetable OGMs. PAT inactivates herbicides exhibiting PPT as an active compound by detoxification of the latter. Detoxification, which results from acetylation of the free amino group present in the PPT, makes the latter incapable of competing in an inhibitory manner with glutamine synthase (GS), thus enabling the removal of the toxic ammonia from the plant cell by conversion of glutamate to glutamine, a reaction that is catalized by the GS (Lindsey, 1992 Molecular cloning of ICAM-3, a third ligand for LFA-1, constitutively expressed on resting leukocytes, Nature 360, 481-484 (3 Dec. 1992) doi:10.1038/360481a0).

Alternatively, the presence of the chimeric gene in transformed cells may be determined by means of other techniques known from the prior art (Sambrook et al “Molecular Cloning, a laboratory manual”, CSHL Press, Cold Spring Harbor, N.Y., 1989), as Southern and PCR.

The techniques to link operatively the components of the inventive recombinant or expression vectors are well known from the prior art and include the use of synthetic ligands containing one or more restriction endonuclease, as transcribed, for instance, in Sambrook et al “Molecular Cloning, a laboratory manual”, CSHL Press, Cold Spring Harbor, N.Y., 1989. Chimeric genes of the present invention may be linked to a vector having at least one replication system, for example, E. coli. Thus after each manipulation the resulting constructs can be cloned and sequenced.

Recombinant and/or expression vectors of the present invention can be used to transform a variety of organisms, including, but not limited to plants. The plants that can be transformed by using recombinant and/or expression vectors of the present invention include monocotyledons angiospermae (for example, grass, maize, grains, oat, wheat and barley . . . ), dicotyledoneous angiosperms (for example Arabidopsis, tobacco, legumes, alfalfa, oats, eucalyptus, maple . . . ), gymnospermae (for example, pine tree, white spruce, Larix . . . ). The plant transformation protocols are already well known from the prior art (Manual de transformação genetic de plantas. Brasília: EMBRAPA-SPI/EMBRAPA-CENARGEM, Chapters 3 and 7, 1998). In a preferred embodiment, the recombinant and/or expression vectors of the present invention are employed to transform dicotyledoneous plants. Preferably the plant is selected from the family of Euphorbiaceae, more preferably of the species Manihot esculenta. Other plants may be transformed in a useful manner with the recombinant and/or expression vector of the present invention, including, but not limited to: Anacardium, Anona, Archis, Artocarpus, Asparagus, Atropa, Avena, Brassica, CArica, Citrus, Citrullus, Capsicum, Carthamus, Cocos, Coffea, Cunimis, Cucurbita, Daucus, Elaeis, Fragaria, Glycine, Gossypium, Helianthus, Heterocallis, Hordeum, Hyoseyamus, Lactuca, Linum, Lolium, Lupinus, Lycopersicon, Malus, Manihot, Majorana, Medicago, Nicotiana, Olea, Oryza, Panieum, Pannesetum, Passiflora, Persea, Phaseolus, Pistachia, Pisum, Pyrus, Prunus, Psidium, Raphanus, Ricinus, Secale, Senecio, Sinapis, Solanum, Sorghum, Theobromus, Trigonella, Triticum, Vicia, Vitis, Vigna, and Zea.

The transcription termination signal and the polyadenylation region of the present invention includes, but is not limited to termination signal of SV40, adenylation signal of HSV TK, termination signal of the gene of A. tumefasciens nopaline synthase (nos), CaMV 35S RNA gene termination signal, termination signal of the virus that attacks Trifolium subterranean (SCSV), termination signal of the Aspergillus nidulans trpC gene of, and other similar ones. Preferably, the terminator used in the present invention is the terminator of the gene Mec1.

The recombinant and/or expression vectors of the present invention may be introduced into the genome of the desired host plant by a number of conventional techniques. For instance, introduction mediated by A. tomefasciens; electroporation; protoplast fusion; injection into reproductive organs; injection into immature embryos; microinjection of protoplasts of plant cells; by using ballistic methods such as bombardment of particles covered with DNA and others. The choice of the technique will depend on the plant to be transformed. For instance, dicotyledons plants and some monocotyledons and gymnospermae can be transformed by plasmid technology Ti of Agrobacterium. Recombinant and/or expression vectors may be combined with appropriate T-DNA flanking regions and introduced into the conventional host vector A. tumefasciens. The function of virulence of the host A. tumefasciens will direct the insertion of the genetic constructs and adjacent marker within the DNA of the plant cell when the cell is infected by the bacterium. Transformation techniques mediated by A. tumefasciens, including disarming and the use of binary vectors, are well described in the scientific literature (as mentioned in patent application US 20020152501, Horsch et al, Science 233:498, 1984; and Fraley et al, Proc. Ntl. Acad. Sci. USA 80:4803, 1983). The present invention may use various binary vectors, among them the binary vector of type pBI 121.

Microinjection techniques are known from the prior art and well described in the scientific and patent literature. The introduction of recombinant and/or expression vectors by using polyethylene glycol precipitations is described in Paszkowski et al. Embo J. 3:2717-2722, 1984 (as mentioned in patent application US20020152501). Electroporation techniques are described in From et al. Proc. Natl. Acad. Sci. USA 82:5824, 1985 (as mentioned in patent application US 20020152501). Ballistic transformation techniques are described in Klein et al Nature 327:70-73, 1987 (as mentioned in patent application US 20020152501). The introduction of the recombinant and/or expression vectors of the present invention may be made in leave tissues, dissociated cells, protoplasts, seeds, embryos, meristematic regions, cotyledons, hypocotyledons, and others. Preferably, the present invention uses the transformation by introduction mediated by A. tumefasciens using A. thaliana as model plant (Clough at al, “Floral dip: a simplified method for Agrobacterium-mediated transformation of A. thaliana”, Plant, J. 1998 December; 16(6):735-43). However, other transformation methods may be used to insert the recombinant and/or expression vectors of the present invention, such as the ballistic one, which consists of a direct transformation technique of the DNA that uses microbullets propelled at high speed to carry the DNA into the cells [Rech, E. L.; Aragão, F. J. L. Biobalística. In: Manual de Transformação Genética de Plantas (Brasileiro, A. C. M. & Carneiro, V. T. C. Eds.), EMBRAPA Serviço de Produção de Informações—SPI. 1998, 106 pp], and via pollinic tube. The transformation method via pollinic tube was disclosed for the first time by Zhou et al (Zhou, G., Wang, J., Zeng, Y., Huang, J., Qian, S., and Liu, G. Introduction of exogenous DNA into cotton embryos. Meth. In Enzymol. 101:433-448, 1983) and consists in applying a DNA solution into the upper part of the young apple after pollination. Using this technique, the exogenous DNA can reach the ovary through the passage left by the pollinic tube and integrate the zygotic cells, already fertilized but not divided.

Once the cells have been transformed, by any of the above-mentioned techniques, the cells having the recombinant and/or expression vector of the present invention incorporated in their genome can be selected by means of a maker, like the marker of resistance to hygromycin or canamicyn. The cells of transformed plants may then be cultivated to regenerate a whole plant that has the transformed genotype and, finally, the desired phenotype. Such regeneration techniques rely on the manipulation of certain phytohormones in a culture medium of tissues, typically containing a biocidal and/or herbicidal marker, which should be introduced together with the desired nucleotide sequence. Regeneration of plants from protoplast culture is described in Evans t al (Evans et al, Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985, as mentioned in patent application US 20020152501). The regeneration may also be obtained through plant callus, explants, organs or parts thereof. Such regeneration techniques are well described in the prior art, such as in Leelavanthi et al [Leelavanthi et al, A simple and rapid Agrobacterium-mediated transformation protocol for cotton (G. hirsutum L.): Embryogenic calli as a source to generate large numbers of transgenic plants, Plant Cell Rep (2004) 22:465-470]. This work describes a protocol for transformation and regeneration of cotton, wherein the embryogenic callus with Agrobacterium is cultured under dehydration stress and antibiotic selection for 3 to 6 months for the regeneration of various transgenic embryos, an average of 75 globular embryos. Observed on the selection of slides, these embryos are cultured and multiplied in the medium, followed by the development of cotyledonary embryos on the medium of maturation of the embryo. In order to obtain an average of 12 plants per Petri dish of calli cultured. About 83% of these plants are transgenic. The resulting transformed plants may be reproduced either in sexual or in asexual manner, by using methods known from the prior art ([Leelavathi et al, A simple and rapid Agrobacterium-mediated transformation protocol for cotton (Gossipium hirsutum L.): Embryogenic calli as a source to generate large numbers of transgenic plants, Plant Cell Rep, 2004, 22: 465-470], to give successive generations of transgenic plants.

The production or RNA in cells may be controlled by selection of the promoter sequence, by selection of the number of functional copies or through the site of integration of polynucleotide incorporated into the host genome. An organism may be transformed by using a recombinant and/or expression vector of the present invention having more than one open reading frame coding from a polypeptide of interest.

The isolated polynucleotide of the present invention is also useful in mapping the genome, in the physical mapping and in positional cloning of genes. The sequence identified as SEQ ID NO1 and its variants may be used for projecting probes and primers of oligonucleotides. The oligonucleotide probes using the polynucleotide of the present invention may be used to detect the presence of the promoter and regulatory regions of the Mec1 gene in any organism having DNA sequences sufficiently similar in their cells using techniques well known from the prior art, like the dot blot DNA hybridization techniques (Sambrook, J., Fritsch, E. F., Maniatis, T. Molecular cloning a laboratory manual, 2^{nd} edition [M]. New York: Cold Spring Harbor Laboratory Press, 1989).

The oligonucleotide primers designed using the polynucleotide of the present invention may be used for PCR amplification. The polynucleotide of the present invention may also be used for labeling or identifying an organism or reproductive material thereof. This label can be obtained, for instance, by stable introduction of a non-functional, non-disruptive heterologous polynucleotide identifier in an organism under the control of the polynucleotide of the present invention.

The polynucleotide proposed for the present invention has been obtained by following preferably these steps:
1—the genetic material of the samples of the possible candidates can be isolated according to any procedure that enables one to have access to the complete genetic material, as for instance, extraction methods that use organic solvents;
2—starting from the isolated material, one carries out a PCR reaction to obtain the fragments. In these reactions specific primers of the selected gene are used, as for example, the gene Mec1. Said primers may be designated as aid of the Primer program (http://frodo.wi.mit.edu/primer3/) (Rozen, S and Skaletsky H. J. 2000 Primer3 on the www for general users and for biologist programmers. In: Krawetz S, Misener S (eds) Bioinformatics Methods and Protocols: Methods in Molecular Biology. Humana Press, Totowa, N.J., pp 365-386) or any other program and process that provides specific primers for the candidates;
3—the PCR reactions may be conducted in a special apparatus for the procedure, like MJ Research thermocycler, PTC-100 model, or any other thermocycler capable of performing its function, in ideal conditions for the reaction, in which one suggests the initial incubation of 92-96° C. for 3-5 minutes, followed by 25-35 cycles (92-96° C. for 30 seconds to 2 minutes for denaturation, 60-65° C. for 30 seconds to 2 minutes for hybridization of the oligonucleotides and 70-75° C. from 15 to 25 minutes for a final extension;
4—the identity of the amplified product may be confirmed, for example, by electrophoretic migration of the fragments, in which the use, for comparison, the positive control constituted by a vector, for example, a vector containing the sequence studied is recommended.

After obtaining the polynucleotide, the transformation process for incorporation of the polynucleotide of the invention will occur. The transformation process should be carried out in the organism of interest through the procedures described before in this specification. The transformation efficiency of the explants should be evaluated through the transient and stable expression of a gene of interest in the tissues of the regenerated propagule, for which the use of the gene gus is recommended. The analysis of expression of the gus gene should be carried out by histochemical assay protocols, for instance, according to adaptation of the protocol described by Jefferson (Jefferson R. A., Kavaaangh T. A. and Bevan M. W. 1987. GUS fusions: β-glucuronidase as a sensitive and versatile gene fusion marker in higher plants. EMBO J. 6: 3901-3907) in leaves, roots, fruits, seeds and flowers (petal, gynoeceum, stamens, pollen) of the transformed plants.

### EXAMPLE

The present invention is further defined in the following examples. It should be understood that these Examples, while indicating a part of the invention, are given only as illustration, without limiting the scope of the present inventions.

Usual techniques of molecular biology such as transformation of bacteria and electrophoresis in agarose gel of nucleic acids are referred to by common terms to describe them. Details of the practice of these techniques, well known from the prior art, are described in Sambrook et al, (Molecular cloning, A Laboratory Manual, 2^{nd} ed (1989), Cold Spring Harbor Laboratory Press). A number of solutions used in experimental manipulation are referred to by their common names like “agarose”, “TBE”, “miniprep”, etc. the compositions of these solutions can be found in reference Sambrook et al (cited above).

### Example 1

### Analysis of the Expression of the Mec1 Gene

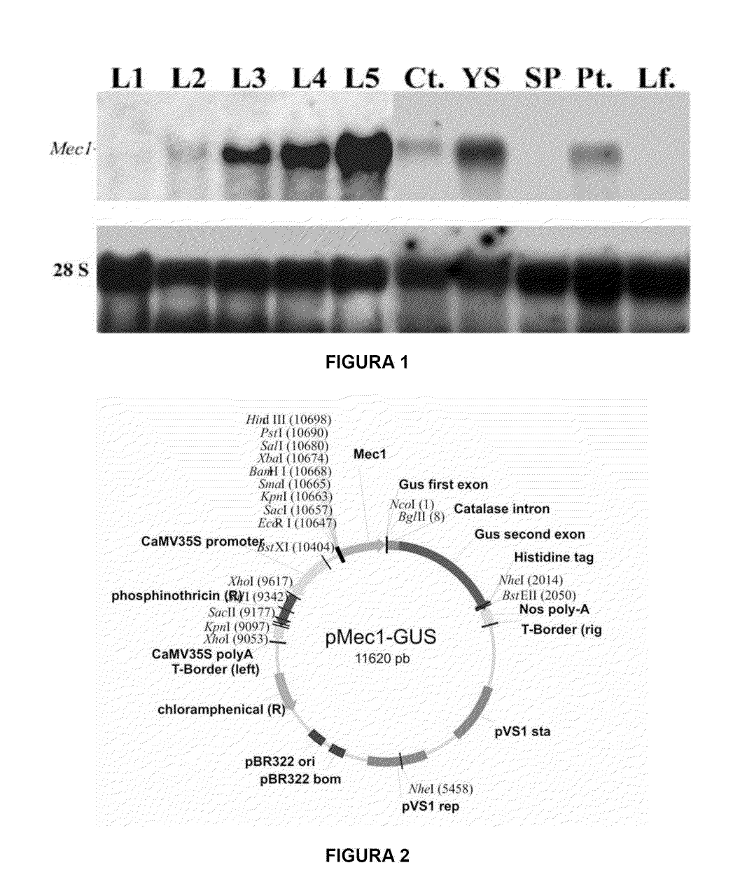

The expression of the Mec1 gene was evaluated in all parts of manioc, which showed a high expression, directed to the storage root. FIG. 1 shows the results of the analysis of the expression in the root, represented by five different layers of tissue (L1 to L5), wherein the expression of the Mec1 gene was greater in L5, constituted by secondary xylem and storage parenchyma. FIG. 1 also shows Mec1 expression signals in the cotyledon (Ct.), in the young stem (YS) and in the petiole (Pt.), but at much lower levels than in the root, while in the leaf (Lf.) and in the stem bark (SP) no expression was detected. These results showed high expression of the Mec1 gene in the root as compared with the other plant parts, wherein in this organ the expression was greater in its central part (layer L5), which is constituted chiefly by vessels (secondary xylem).

### Example 2

### Obtainment of the Vegetable Material

Manioc (Manihot esculenta Crantz) leaves were kindly supplied by Dr. Eloisa Cardoso of company Embrapa Amazônia Oriental (EMBRAPA-CPATU, Belém, PA, Brasil).

### Example 3

### Isolation of the Promoter Sequence by Chain Reaction of the Reversed Polymerase

The genomic DNA was isolated from manioc leaves through Purelink total plant DNA purification kit and quantified by using a Qubit fluorimeter, both supplied by Invitrogen Life Technologies, following the instructions of the manufacturer.

Both samples containing about 10 μg of genomic DNA were totally and separately digested with HaeIII, DraI and HphI restriction enzymes. After extraction with phenol: chloroform: isoamyl alcohol (24:24:1), and precipitation of the ethanol, DNA fragments were self-circularized by T4 DNA ligase and used in the chain reaction of reverse polymerase (PCR). Two reverse primers (Mec2-R: 5′ actggctctgcttccttgggctcttc 3′ (SEQ ID NO: 3) and MEC3-R: 5′ tcctcaggaagtgcagtctgtgctgt 3′ (SEQ ID NO: 4)) and a “forward” primer (Mec4 F: 5′ gctgatgatgctccggctgaagtagc 3′ (SEQ ID NO: 5)) were projected in accordance with the cDNA Mec1 sequence isolated before and registered at the NCBI GenBank (access AY101376).

DNA fragments were amplified by using Mec2-R/Mec4-F primers in the primary reversed PCR and Mec3-R/Mec4-F primers in the secondary reverse PCR. The conditions used in the primary and secondary assays of reverse PCR were: 5 min at 94° C., 30 amplification cycles (1 min at 94° C., 1 min at 03° C. and 1.5 minute at 72° C.) and 20 min at 72° C. for a final expression. The PCR tests were carried out by using the polimerase Advantage 2 kit mix supplied by Clontech (Palo Alto, USA). The amplified products were purified from an agarose gel by using the QIAquick Spin kit (Qiagen) and cloned in the pGEMTeasy vector (Promega Corporation).

PCR assays using Mec9-Primers (5′ ggtgatgagaagagagactatttcgttgaca 3′ (SEQ ID NO: 6)) and Mec11-R (5′ tacctcagcagtagccatagtcagcca 3′ (SEQ ID NO: 7)) were carried out to obtain a contiguous promoter sequence, which was amplified from a non-digested genomic DNA and cloned in the pGEMTeasy vector, generating the pMec1 plasmid. All the clones were sequenced by using a MegaBACE 1000 sequencer (GE Healthcare Life Sciences).

The isolated DNA sequence (SEQ ID NO1), object of this patent document is constituted by 1.135 nucleotides. The DNA sequence (SEQ ID NO1) is constituted by: (1) promoter region exhibiting 875 nucleotides (1-875), (2) non-translated sequence exhibiting 77 nucleotides (876-952), (3) first exon exhibiting 15 nucleotides (953-967), (4) first intron exhibiting 136 nucleotides (968-1103), (5) partial sequence of the second exon exhibiting 32 nucleotides (1004-1135).

The Mec1 gene promoter region exhibits 875 nucleotides and contains a few storage elements. Among them, (1) the TATA Box involved in the formation of the basal apparatus of the transcription, located 103 nucleotides at position upstream of the starting ATG of the translation; (2) ATATT elements that impart expression in the root.

### Example 4

### Analysis of the Sequence

Nucleotide sequences were aligned by using the BLAST algorithm (Altschul S F, Madden T L, Schaffer A A, Zhang J, et al (1997). Gapped BLAST and PSI-BLAST: a new generation of protein database search programs. Nucleic acids Res. 25: 3389-3402 and the ClustalW program (Thompson et al, 1994). The TFSearch program was used to search for putative link sites of transcription factors (Heinemeyer T, Wingender E, Reuter I, HermJakob H, et al (1998). Database on transcriptional regulation: TRANSFAC, TRRD and COMPEL. Nucleic Acids Res. 26: 362-367). PlantCare and PLACE databanks were used for determining cis-acting regulatory elements (Prestridge D S (1991). SIGNAL SCAN: a computer program that scans DNA sequences for eukaryotic transcriptional elements. Comput. Appl. Biosci. 7: 203-206; Higo K, Ugawa Y, Iwamoto M and Korenaga T (1999). Plant cis-acting regulatory DNA elements (PLACE) database: 1999. Nucleic Acids Res. 27: 297-300; Lescot M, Dehais P, Thijs G, Marchal K, et al (2002). PlantCARE, a database of plant cis-acting regulatory elements and a portal to tools for in silico analysis of promoter sequences. Nucleic Acids Rs. 30-3250-327).

### Example 5

### Constructs Used in Experiments for Transient Expression

The 800 pb fragment containing the 35S promoter was released from the pCAMBIA 3201 plasmid (CAMBIA, Canberra, Australia) by digestion with BamHI and NcoI. The F-Mec12 primers (agggatccggtgatgagaagagagactatttcg (SEQ ID NO: 8)) and Mec13-R (cagtagccgatggtcagcca (SEQ ID NO: 9)) containing the BaMHI and NcoI sites (underlined) were used to amplify the Mec1 promoter of 970-bp from the pMec1 plasmid. After digestion with these two enzymes, the 952-pb fragment was cloned between the BamHI and NcoI pCAMBIA 3201 sites, replacing the CaMV 35S promoter, which generated the pCMABIA-Mec1 plasmid. As negative control, the 800-pb fragment containing the 35S promoted was released from the pCAMBIA 3201 plasmid by digestion with BamHI and NcoI, and the DNA fragment of the vector was then self-circularized by T4 DNA ligase. As positive control, one used pCAMBIA 3201.

### Example 6

### Bombardment of Embryonary Bean Shafts

The preparation of the explants and particle bombardment were carried out according to methods described before (Aragao F J L, Barros L M G, Brasileiro A C M, Ribeiro S G, et al (1996). Inheritance of foreign genes in transgenic bean (Phaseolus vulgaris L.) co-transformed via particle bombardment. Theor. Appl. Genet. 93: 142-150). Embryonary bean shafts were separately bombarded superficially and transversely with three plasmids: pCAMBIA-Mec1, pCAMBIA 3201 and pCAMBIA 3201 without the CaMV 35S promoter. After the bombardment (50-mmol·m⁻²s⁻¹), the explants were cultured for 24 hours at 28° C. with photoperiod of 16 h in a MS medium. The tissues were analyzed in situ location of the GUS activity according to the methods described before (Jefferson R A, Kavanagh T A and Bevan M W (1987). GUS fusion: beta-glucuronidase as a sensitive and versatile gene fusion marker in higher plants. EMBO J. 6:3901-3907).

### Example 7

### Analysis of the Function of the Mec1 Gene Promoter Region

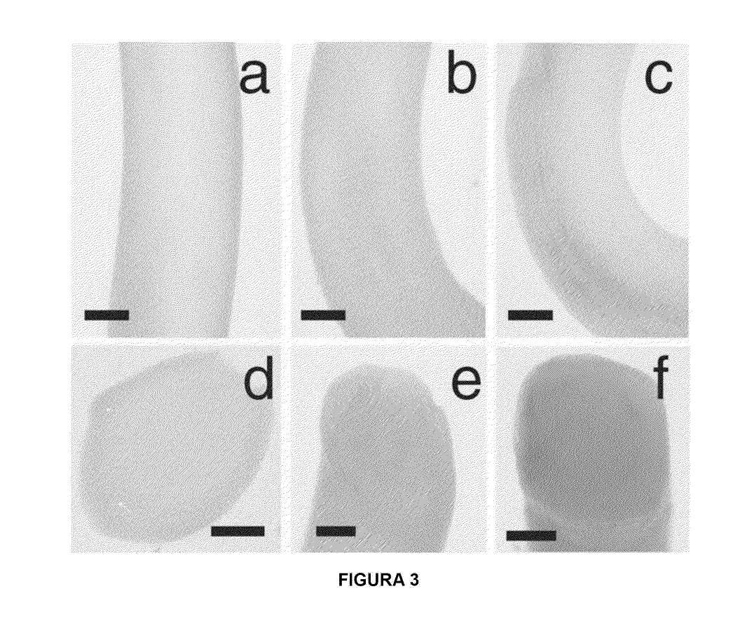

The functionality of the Mec1 gene promoter region was evaluated through genetic transformation experiments. For this purpose, initially the genetic construction pMec1-GUS was made, shown in FIG. 2, in which the Mec1 gene promoter region (1) was placed at a position adjacent the reporter gene of the glucoronidase (GUS)enzyme (2). Then, the pMec1-GUS construct was introduced into bean embryos by bombardment, in which the GUS activity was detected by observing the bluish coloration (FIG. 3: b, e, f), thus proving the functionality of the isolated promoter sequence. FIG. 3 also shows embryos bombarded with the pCAMBIA 3201 vector, without the cauliflower mosaic virus 35S promoter (negative control) (a,d), in which no GUS activity was observed, and embryo bombarded with the pCAMBIA 3201 vector containing the 35S promoter (positive control) (c), in which GUS activity was observed. Comparing the GUS expression standard directed by the two promoters, one can observe that 35S directed the expression to the epidermal cells, while Mec1 directed it to the central part of the embryo, which corresponds to the vascular system that is being formed.

Thus, the isolated nucleotide sequence (SEQ ID NO1) of the present invention, exhibits proven functionality and can be used in vegetable genetic improvement programs.

1. A polynucleotide with tissue-specific activity, characterized by comprising a sequence selected from the group consisting of:
a) sequences that are substantially similar to SEQ ID NO1;
b) complements of the sequence described in SEQ ID NO1;
c) reverse complements of the sequence the sequence described in SEQ ID NO1;
d) reverse sequences of the sequence described in SEQ ID NO1.

2. A chimeric gene characterized by comprising:
a) A polynucleotide of which the sequence is substantially similar to SEQ ID NO1, optionally linked to sequences that enhance expression or promoters of interest, operatively linked to:
b) A sequence of a polynucleotide of interest.

3. A chimeric gene according to claim 2, characterized in that the sequence of polynucleotide of interest may be a coding region or a non-coding region.

4. A chimeric gene according to claim 3, characterized in that the coding region is isolated from an endogenous or heterologous gene.

5. A chimeric gene according to claim 2, characterized in that the polynucleotide sequence of interest may be on the sense or antisense orientation.

6. A chimeric gene according to claim 2, characterized in that the expression enhancing sequences are selected from the group consisting of SV40, HSV-1, AMV, HPV-16, among others.

7. A recombinant vector characterized by containing a chemeric gene according to claim 2.

8. A recombinant vector characterized by comprising:
a) A polynucleotide of which the sequence is substantially similar to SEQ ID NO1, optionally linked to expression enhancing sequences or promoters of interest, operatively linked to:
b) a polynucleotide sequence of interest; and
c) a termination sequence.

9. A recombinant vector according to claim 8, characterized in that the polynucleotide sequence of interest may be a coding region or a non-coding region.

10. A recombinant vector according to claim, characterized ion that the polynucleotide sequence of interest is isolated from an endogenous or heterologous gene.

11. A recombinant vector according to claim 8, characterized in that the termination sequence is selected from the group consisting of SV40 termination signal, HSV TK adenylation signal, Agrobacterium tumefasciens nopaline synthase (NOS) gene termination signal, octopine synthase gene termination signal, CaMV 19S and 35S gene termination signal, maize alcohol dehydrogenase gene termination signal, manopine synthase gene termination signal, beta-phaseolin gene termination signal, ssRUBISCO gene termination signal, sucrose synthase gene termination signal, termination signal of the virus that attacks the Trifolium substerranean (SCSV), Aspergillus nidulans trpC gene termination signal and other similar ones.

12. A recombinant vector according to claim 8, characterized in that the expression enhancing sequences are selected from the group consisting of SV40, HSV-1, AMV, HPV-16, among others.

13. A transformed cell characterized by comprising a recombinant vector according to any one of claims 7 to 12.

14. A plant, or a part thereof, or a propagule or progeny thereof, characterized by comprising a recombinant vector according to any one of claims 7 to 12.

15. A method for modifying the expression of genes in one organism, characterized by incorporating, in a stable manner, into the genome of the organism, a recombinant vector according to any one of claims 7 to 12, or a chimeric gene according to any one of claims 2 to 6.

16. A method according to claim 15, characterized in that the organism is a plant.

17. A method for producing a plant having the expression of a gene modified, characterized by comprising the following steps:
a) transforming a plant cell, tissue, organ or embryo with a recombinant vector according to any one of claims 7 to 12, or a chimeric gene according to any one of claims 2 to 6;
b) selecting transformed cells, cell callus, embryos or seeds;
c) regenerating mature plants of transformed cells, callus cells, embryos or seeds selected in step (b);
d) selecting mature plants of step (c) having the expression of the gene modified as compared with a non-transformed plant.
